(19) United States
(12) Patent Application Publication
(10) Pub. No.: US 2024/0215835 A1
(43) Pub. Date: Jul. 4, 2024

(54) NON-CONTACT BLOOD VESSEL ANALYZING METHOD

(71) Applicant: LEIMAC LTD., Moriyama-shi, Shiga (JP)

(72) Inventors: Takunori SHIMAZAKI, Kawanishi (JP); Yoshifumi KAWAKUBO, Awa (JP); Yuhei HAYASHI, Moriyama (JP); Jun MITSUDO, Moriyama (JP); Jumpei OHUCHI, Moriyama (JP)

(21) Appl. No.: 18/556,935

(22) PCT Filed: Feb. 26, 2022
(86) PCT No.: PCT/JP2022/008098

§ 371 Date: Oct. 24, 2023

(30) Foreign Application Priority Data
May 21, 2021 (JP) 2021-085767
Jan. 25, 2022 (JP) 2022-009552

(51) Int. Cl. A61B 5/02; A61B 5/00

(52) U.S. Cl. CPC A61B 5/02007; A61B 5/0077

(57) ABSTRACT

Provided is a non-contact blood vessel analyzing method that enables speedy and detailed contactless analysis of states of blood vessels. This non-contact blood vessel analyzing method uses a non-contact blood vessel analyzing device 1 that includes a light irradiator 2 that casts light on a skin surface site including a blood vessel region, an image acquirer 3 that acquires an image 3im that is a moving image or a series of still images of the skin surface site, and an image processor 4 that derives at least a first waveform that indicates temporal change of a brightness value in a first wavelength domain and a second waveform that indicates temporal change of a brightness value in a second wavelength domain, in a certain region in the image 3im. A frequency, a phase, or an amplitude, of the first waveform and the second waveform, is derived. For comparison, further, the frequency, the phase, or the amplitude of the first waveform and the second waveform in the certain region are derived after a certain amount of time elapses, and/or the frequency, the phase, or the amplitude of the first waveform and the second waveform are derived in a region that is different from the certain region.

### TECHNICAL FIELD

The present invention relates to a non-contact blood vessel analyzing method that enables states of blood vessels to be analyzed contactlessly.

### BACKGROUND ART

It is not unusual for states of blood vessels to be analyzed using various types of analyzing devices, besides general visual examination, palpation, and so forth. Among blood vessel analyzing methods using such analyzing devices, there is known a blood vessel analyzing method in which light of a plurality of wavelength domains is cast on a skin surface site including a blood vessel region that is an object, and images are analyzed, as disclosed in WO 2017/051455. Such blood vessel analyzing methods in which images are analyzed are non-contact blood vessel analyzing methods in which analysis is performed without touching the human body or the like (i.e., contactlessly), and accordingly enable infection risk to be suppressed and also speedy analysis to be performed.

### BRIEF SUMMARY

However, the blood vessel analyzing method according to WO 2017/051455 goes no further than to impart contrast to an image of blood vessels with different depths from the skin by using a plurality of wavelength domains, and further development as a non-contact blood vessel analyzing method is possible.

The present invention has been made in light of the foregoing situation, and accordingly it is an object thereof to provide a non-contact blood vessel analyzing method that enables speedy and detailed contactless analysis of states of blood vessels.

In order to achieve the above object, a non-contact blood vessel analyzing method according to an embodiment of the present invention, using a non-contact blood vessel analyzing device that includes a light irradiator that casts light on a skin surface site including a blood vessel region, an image acquirer that acquires an image that is a moving image or a series of still images of the skin surface site, and an image processor that derives at least a first waveform that indicates temporal change of a brightness value in a first wavelength domain and a second waveform that indicates temporal change of a brightness value in a second wavelength domain, in a certain region in the image, includes deriving a frequency, a phase, or an amplitude, of the first waveform and the second waveform, and for comparison, further deriving the frequency, the phase, or the amplitude of the first waveform and the second waveform in the certain region after a certain amount of time elapses, and/or deriving the frequency, the phase, or the amplitude of the first waveform and the second waveform in a region that is different from the certain region.

Preferably, a blood flow velocity is derived from the phase of the first waveform in the certain region and the phase of the first waveform in the region that is different from the certain region.

Another non-contact blood vessel analyzing method according to the embodiment of the present invention, using a non-contact blood vessel analyzing device that includes a light irradiator that casts light on a skin surface site including a blood vessel region, an image acquirer that acquires an image that is a moving image or a series of still images of the skin surface site, and an image processor that derives a heightwise form of a surface in a certain region in the image by an optical 3D surface profiling technique, includes deriving a height, a cross-sectional area, or a volume, of a blood vessel, from the heightwise form.

Preferably further included is, for comparison, deriving the height, the cross-sectional area, or the volume, of the blood vessel in the certain region, after a certain amount of time elapses, and/or deriving the height, the cross-sectional area, or the volume, of the blood vessel in a region that is different from the certain region.

Yet another non-contact blood vessel analyzing method according to the embodiment of the present invention, using a non-contact blood vessel analyzing device that includes a light irradiator that casts light on a skin surface site including a blood vessel region, an image acquirer that acquires an image that is a moving image or a series of still images of the skin surface site, and an image processor that derives a heightwise form of a surface in a certain region in the image by an optical 3D surface profiling technique, and also derives a first region waveform and a second region waveform that indicate temporal change of a brightness value in a first region and a second region in the image, includes deriving a cross-sectional area of a blood vessel from the heightwise form, deriving a blood flow velocity from a phase of the first region waveform and a phase of the second region waveform, and deriving a volume of blood flow from the cross-sectional area and the blood flow velocity.

According to the non-contact blood vessel analyzing method of the present invention, speedy and detailed contactless analysis of states of blood vessels can be realized.

### BRIEF DESCRIPTION OF THE DRAWINGS

FIG. 1 is a schematic diagram illustrating a usage example of a non-contact blood vessel analyzing device used in a non-contact blood vessel analyzing method according to an embodiment of the present invention.

FIG. 2 is a schematic diagram illustrating an image processor realized by a computer system in the non-contact blood vessel analyzing device 1 illustrated in FIG. 1.

FIG. 3 is a photograph that shows an analysis example, in the above usage example, showing a skin surface site of a finger.

FIG. 4 shows an analysis example, in the above usage example, showing a first waveform and a second waveform in a certain region of the skin surface site shown in FIG. 3.

FIG. 5 is a schematic diagram illustrating an internal structure beneath the skin.

FIG. 6 is a photograph that shows an analysis example, in the above usage example, showing a skin surface site of an arm.

FIG. 7 shows an analysis example, in the above usage example, showing a first waveform in two regions of the skin surface site shown in FIG. 6.

FIG. 8 is a photograph showing three-dimensional image derived using a photometric stereo method in the above usage example.

FIG. 9 is a graph that shows an analysis example, in the above usage example, showing a heightwise form taken along a certain line segment transecting a blood vessel derived using the photometric stereo method.

FIG. 10 is a photograph showing a location of the certain line segment of the graph shown in FIG. 9.

### DETAILED DESCRIPTION

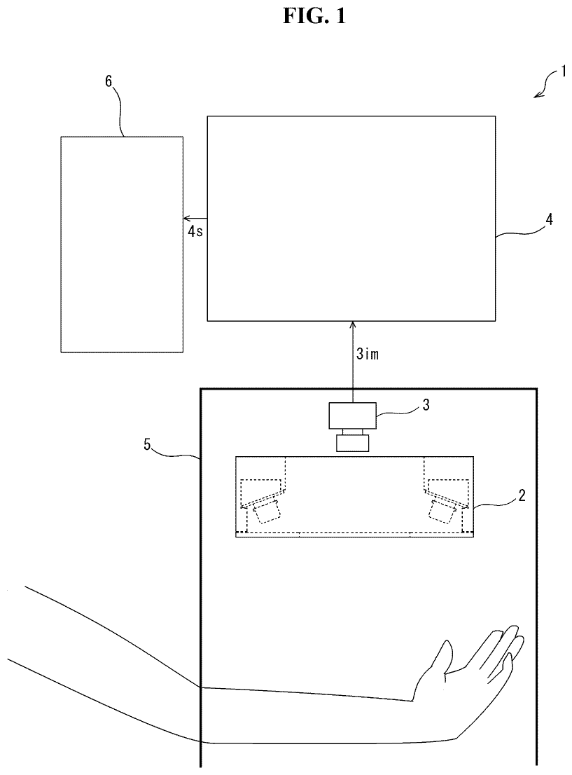

Embodiments for carrying out the present invention will be described below. A non-contact blood vessel analyzing device 1 used in a non-contact blood vessel analyzing method according to an embodiment of the present invention includes a light irradiator 2, an image acquirer 3, and an image processor 4, as illustrated in FIG. 1.

The light irradiator 2 casts light onto a skin surface site including a blood vessel region. The skin surface site including the blood vessel region is not limited in particular, and can be an arm, fingers, or the like, such as illustrated in FIG. 1, for example, and also include vascular access. The light irradiator 2 can include a light-shielding box 5 that shields external light. Note that a ring illuminator is illustrated in FIG. 1 as an example of the light irradiator 2.

The light irradiator 2 emits light of at least a first wavelength domain and light of a second wavelength domain. This enables a first waveform indicating temporal change of brightness values of the first wavelength domain in the image, and a second waveform indicating temporal change of brightness values of the second wavelength domain therein, to be derived at the image processor 4, which will be described later. For example, light of the first wavelength domain is green light, and light of the second wavelength domain is red light. Also, the light irradiator 2 is capable of emitting light of other wavelength domains, such as light of a third wavelength domain, and a third waveform or the like, indicating temporal change of brightness values of another wavelength domain such as the third wavelength domain or the like, in the image, can be derived at the image processor 4. For example, light in the third wavelength domain is blue light.

Note that the first waveform and the second waveform can be derived at the image processor 4 by emitting white light from the light irradiator 2, besides emitting the light of the first wavelength domain and the light of the second wavelength domain from the light irradiator 2, and providing a color filter upstream of the image acquirer 3, through which the light of the first wavelength domain is passed to derive the first waveform, and through which the light of the second wavelength domain is passed to derive the second waveform.

The image acquirer 3 preforms image-capturing of the skin surface site including the blood vessel region on which light is cast by the light irradiator 2, thereby acquiring an image 3im that is a moving image or a series of still images.

The image processor 4 derives at least the first waveform indicating temporal change of brightness values of the first wavelength domain in a certain region in the image 3im acquired by the image acquirer 3, and the second waveform indicating temporal change of brightness values of the second wavelength domain therein.

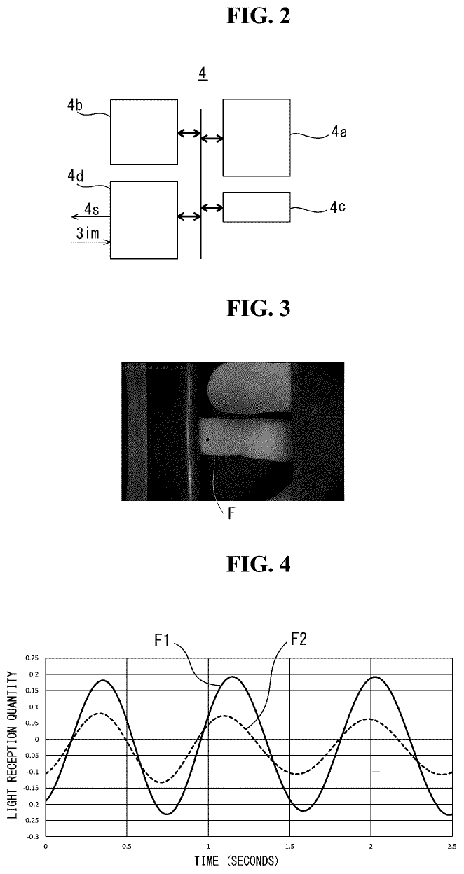

The image processor 4 normally is realized by a computer system as illustrated in FIG. 2, and has a program for performing image processing in program memory 4a. In FIG. 2, sign 4b denotes a CPU, sign 4c denotes work memory, and sign 4d denotes other portions including an input/output unit.

The non-contact blood vessel analyzing method that enables states of blood vessels to be analyzed contactlessly in detail, using the non-contact blood vessel analyzing device 1 described above, will be described below.

From the first waveform and the second waveform are derived a frequency, a phase, or an amplitude thereof. Thereafter, the frequency, the phase, or the amplitude in the certain region is further derived in the certain region after a certain amount of time elapses, and the frequency, the phase, or the amplitude in the certain region and the frequency, the phase, or the amplitude in the certain region after a certain amount of time elapses are compared, which enables the states of the blood vessels to be analyzed. Also, further deriving the frequency, the phase, or the amplitude in a region that differs from the certain region, and comparing the frequency, the phase, or the amplitude of the first waveform and the second waveform in the certain region with the frequency, the phase, or the amplitude in a region different from the certain region enables the states of the blood vessels to be analyzed.

For example, as shown in FIG. 3, light (green light and red light) is cast from the light irradiator 2 to a skin surface site of a finger, and image-capturing is performed by the image acquirer 3 to acquire the image 3im that is a moving image or a series of still images. Thereafter, as shown in FIG. 4, the first waveform (waveform indicted by solid line and denoted by sign F1 in FIG. 4) corresponding to the green light, and the second waveform F2 (waveform indicted by dashed line and denoted by sign F2 in FIG. 4) corresponding to the red light, are derived by the image processor 4 in a certain region (denoted by sign F in FIG. 3) in the image 3im. Note that the vertical axis in FIG. 4 is light reception quantity prior to correlation with actual brightness values, which is a relative value, and accordingly no unit has been listed.

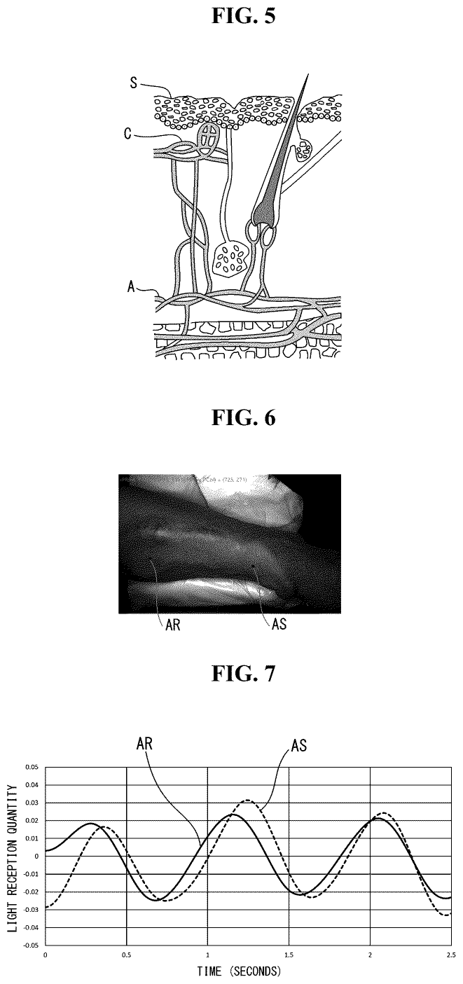

Inside the human body, as illustrated in FIG. 5, capillary blood vessels (denoted by sign C in the drawings) are formed below the skin (denoted by sign S in the drawings), and therebelow are formed arterioles (denoted by sign A in the drawings) and so forth. Light components with short wavelengths (e.g., green) reach blood vessels at shallow portions from the skin (capillary blood vessels or the like) and are reflected, while light components with long wavelengths (e.g., red) reach blood vessels at deep portions from the skin (arterioles or the like) and are reflected.

For example, an arrangement may be made in which the first waveform and the second waveform are derived prior to surgery and after surgery is started, and the states of the blood vessels can be analyzed regarding whether the frequency, the phase, or the amplitude of the first waveform and the second waveform changes after the surgery is started (i.e., after a certain amount of time has elapsed), from that of the first waveform and the second waveform in the same certain region. This can be applied to the anesthesia management and so forth. Note that it is conceivable that deterioration in peripheral circulation will become markedly manifested at an early stage in the capillary blood vessels or the like, which are at shallow portions from the skin, and accordingly change in the first waveform corresponding to the green light will become markedly manifested at an early stage. Note that the skin surface site to be analyzed is not limited to the skin surface sites of the fingers.

Also, for example, as shown in FIG. 6, light (green light and red light) is cast from the light irradiator 2 to a skin surface site of an arm, and image-capturing is performed by the image acquirer 3 to acquire the image 3im that is a moving image or a series of still images. Thereafter, the first waveform corresponding to the green light and the second waveform corresponding to the red light are respectively derived by the image processor 4 in two regions that are separated from each other by a predetermined distance (i.e., a certain region, and a region that is different therefrom) (denoted by signs AR and AS in FIG. 6) in the image 3im. FIG. 7 shows the first waveform. In FIG. 7, the phase of the first waveform is shifted among two regions. The blood flow velocity can be found from this phase difference and the distance between the two regions. This analysis of the blood flow velocity is applicable to the analysis regarding prior to surgery and after surgery is started, which is described above. Note that the skin surface site to be analyzed is not limited in particular to the skin surface site of the arm. Also, the vertical axis in FIG. 7 is the light reception quantity prior to correlation with actual brightness values, which is a relative value (unrelated to the value on the vertical axis in FIG. 4 as well), and accordingly no unit has been listed.

Next, a non-contact blood vessel analyzing method that uses an optical 3D surface profiling technique will be described. In this non-contact blood vessel analyzing method, the light irradiator 2 casts light on the skin surface site including the blood vessel region, and the image acquirer 3 preforms image-capturing, thereby acquiring an image 3im that is a series of still images (or a moving image), in the same way as that above. The heightwise form of the surface in a certain region in the image 3im is then derived by the image processor 4. The photometric stereo method and fringe projection method are representative examples of the optical 3D surface profiling technique.

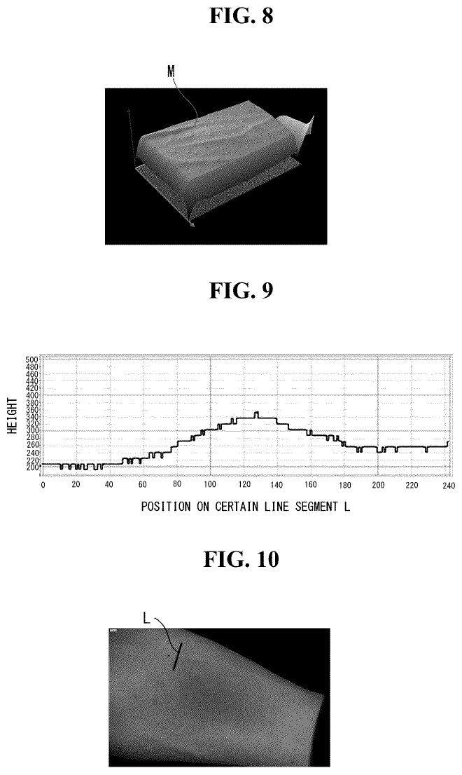

A plurality of light irradiators 2 (e.g., eight) are necessary in the non-contact blood vessel analyzing method using the photometric stereo method, since light is cast on the skin surface site from different directions. Performing processing with the image processor 4 using the photometric stereo method enables images expressing the three-dimensional form of the skin surface site by brightness values to be obtained, as denoted by sign M in FIG. 8. Also, as shown in FIG. 9, the heightwise form of surface thereof can be derived, with a certain line segment transecting one blood vessel in the image (denoted by sign L in FIG. 10), as shown in FIG. 10, as the certain region. Note that the vertical axis in FIG. 9 is that prior to correlation between brightness values and physical height, and accordingly no unit has been listed. Also, the horizontal axis in FIG. 9 is that prior to correlation with values of physical positions, and accordingly no unit has been listed.

Height, cross-sectional area, or volume of the blood vessel can be derived from the heightwise form of the surface in the certain region. This enables the height, the cross-sectional area, or the volume of the blood vessel to be further derived in the certain region after a certain amount of time has elapsed, and comparing the height, the cross-sectional area, or the volume of the blood vessel in the certain region with the height, the cross-sectional area, or the volume of the blood vessel in the certain region after a certain amount of time has elapsed enables the states of the blood vessel to be analyzed. Also, further deriving the height, the cross-sectional area, or the volume of the blood vessel in a region that is different from the certain region, and comparing the height, the cross-sectional area, or the volume of the blood vessel in the certain region with the height, the cross-sectional area, or the volume of the blood vessel in the region that is different from the certain region, enables the states of the blood vessel to be analyzed. Thus, fatigue of the blood vessel (e.g., vein or surgical anastomosis) and so forth can be analyzed.

Next, a non-contact blood vessel analyzing method that uses the optical 3D surface profiling technique and the above analysis technique for blood flow velocity in combination. In this non-contact blood vessel analyzing method, the light irradiator 2 casts light on the skin surface site including the blood vessel region, and the image acquirer 3 preforms image-capturing, thereby acquiring an image 3im that is a series of still images (or a moving image), in the same way as described above. The heightwise form of the surface in a certain region in the image 3im is then derived by the image processor 4. A first region waveform and a second region waveform that indicate temporal change of brightness values in a first region and a second region in the image 3im are also derived by the image processor 4.

Now, the certain region in the image 3im can be a certain line segment transecting one blood vessel, such as denoted by the sign L in FIG. 10. Also, the first region and the second region in the image 3im can be two regions that are separated by a predetermined distance, such as denoted by the signs AR and AS in FIG. 6.

The cross-sectional area of the blood vessel is derived from the heightwise form of the surface in the certain region. Now, an arrangement can be made in which the thickness of the walls and so forth of the blood vessel are subtracted. Also, the blood flow velocity is derived from the phase of the first region waveform and the phase of the second region waveform. More specifically, the blood flow velocity is derived from the phase difference therebetween and the distance between the two regions (the first region and the second region). The volume of blood flow is then derived from the cross-sectional area and the blood flow velocity.

Note that the non-contact blood vessel analyzing device 1 can include a display device 6 (see FIG. 1), with display data 4s from the image processor 4 being input to the display device 6, such that graphs and the like such as shown in FIG. 4, FIG. 7, and FIG. 9 are selectively or simultaneously displayed.

The entire non-contact blood vessel analyzing device 1 can be integrated, and a terminal equipped with a camera, such as a smartphone, a laptop computer, or the like, for example, can be used.

The non-contact blood vessel analyzing method using the non-contact blood vessel analyzing device 1 in this way enables contactless analysis of the states of blood vessels, thereby enabling infection risk to be suppressed and also speedy and detailed analysis to be performed, without any need for work that affects the human body, such as injecting contrast dye or the like. Also, the human body can be monitored and analyzed at various locations and over a broad field of view.

Although the non-contact blood vessel analyzing method according to an embodiment of the present invention has been described above, the present invention is not limited to the description in the embodiment described above, and various design modifications can be made within the scope of matters set forth in the Claims.

### REFERENCE SIGNS LIST

1 Non-contact blood vessel analyzing device
2 Light irradiator
3 Image acquirer
3im Image
4 Image processor
4a Program memory
4b CPU
4c Work memory
4d Other portions of computer system
4s Display data
5 Light-shielding box
6 Display device

1.-5. (canceled)

6. A non-contact blood vessel analyzing method using a non-contact blood vessel analyzing device that includes
a light irradiator that emits light of a first wavelength domain and light of a second wavelength domain of which a depth of penetration from skin differs from that of the first wavelength domain, for being cast on a skin surface site including a vascular region,
an image acquirer that acquires an image that is a moving image or a series of still images of the skin surface site, and
an image processor that derives a first waveform that indicates temporal change of a brightness value in the first wavelength domain and a second waveform that indicates temporal change of a brightness value in the second wavelength domain, in a certain region in the image,
the non-contact blood vessel analyzing method comprising:
deriving a frequency, a phase, or an amplitude, of the first waveform and the second waveform; and
for comparison, further deriving the frequency, the phase, or the amplitude of the first waveform and the second waveform in the certain region after a certain amount of time elapses, and/or deriving the frequency, the phase, or the amplitude of the first waveform and the second waveform in a region that is different from the certain region.

7. A non-contact blood vessel analyzing method using a non-contact blood vessel analyzing device that includes
a light irradiator that casts light on a skin surface site including a vascular region,
an image acquirer that acquires an image that is a moving image or a series of still images of the skin surface site through a color filter for transmitting light of a first wavelength domain and light of a second wavelength domain of which a depth of penetration from skin differs from that of the first wavelength domain, and
an image processor that derives a first waveform that indicates temporal change of a brightness value in the first wavelength domain and a second waveform that indicates temporal change of a brightness value in the second wavelength domain, in a certain region in the image,
the non-contact blood vessel analyzing method comprising:
deriving a frequency, a phase, or an amplitude, of the first waveform and the second waveform; and
for comparison, further deriving the frequency, the phase, or the amplitude of the first waveform and the second waveform in the certain region after a certain amount of time elapses, and/or deriving the frequency, the phase, or the amplitude of the first waveform and the second waveform in a region that is different from the certain region.

8. The non-contact blood vessel analyzing method according to claim 6, wherein
a blood flow velocity is derived from the phase of the first waveform in the certain region and the phase of the first waveform in the region that is different from the certain region.

9. The non-contact blood vessel analyzing method according to claim 7, wherein
a blood flow velocity is derived from the phase of the first waveform in the certain region and the phase of the first waveform in the region that is different from the certain region.

10. A non-contact blood vessel analyzing method using a non-contact blood vessel analyzing device that includes
a light irradiator that casts light on a skin surface site including a vascular region,
an image acquirer that acquires an image that is a moving image or a series of still images of the skin surface site, and
an image processor that derives, with a certain line segment that transects one blood vessel as a certain region, a heightwise form of a surface in the certain region in the image by an optical 3D surface profiling technique,
the non-contact blood vessel analyzing method comprising:
deriving a height, a cross-sectional area, or a volume, of the blood vessel, from the heightwise form.

11. The non-contact blood vessel analyzing method according to claim 10, further comprising:
for comparison, deriving the height, the cross-sectional area, or the volume, of the blood vessel in the certain region, after a certain amount of time elapses, and/or deriving the height, the cross-sectional area, or the volume, of the blood vessel in a region that is different from the certain region.

12. A non-contact blood vessel analyzing method using a non-contact blood vessel analyzing device that includes
a light irradiator that casts light on a skin surface site including a vascular region,
an image acquirer that acquires an image that is a moving image or a series of still images of the skin surface site, and
an image processor that derives a heightwise form of a surface in a certain region in the image by an optical 3D surface profiling technique, and also derives a first region waveform and a second region waveform that indicate temporal change of a brightness value in a first region and a second region in the image,
the non-contact blood vessel analyzing method comprising:
deriving a cross-sectional area of a blood vessel from the heightwise form, deriving a blood flow velocity from a phase of the first region waveform and a phase of the second region waveform, and deriving a volume of blood flow from the cross-sectional area and the blood flow velocity.
